(19) United States
(12) Patent Application Publication
(10) Pub. No.: US 2014/0342402 A1
(43) Pub. Date: Nov. 20, 2014

(54) Stabilizing RNA by Incorporating Chain-Terminating Nucleosides at the 3'-Terminus

(71) Applicant: Board of Supervisors of Louisiana State University and Agricultural and Mechanical College, Shreveport, LA (US)

(72) Inventors: Robert E. Rhoads, Shreveport, LA (US); Wei Su, Seattle, WA (US); William F. Marzluff, Hillsborough, NC (US)

(73) Assignee: Board of Supervisors of Louisiana State University and Agricultural and Mechanical College, Batou Rouge, LA (US)

(21) Appl. No.: 14/363,316

(22) PCT Filed: Jan. 3, 2013
(86) PCT No.: PCT/US13/20059

§ 371 Date: Jun. 6, 2014

Related U.S. Application Data
(60) Provisional application No. 61/583,043, filed on Jan. 4, 2012.
(60) Provisional application No. 61/583,043, filed on Jan. 4, 2012.

(51) Int. Cl. C12P 21/00; C12N 15/87

(52) U.S. Cl. CPC C12P 21/00; C12N 15/87
USPC 435/69.1; 536/23.1

(57) ABSTRACT

A method is disclosed for stabilizing histone stem-loop-containing mRNA by the addition of a chain-terminating nucleoside. The novel, synthetic mRNA contains a 3′ histone stem-loop sequence. At the 3′ end of the mRNA a chain-terminating nucleoside is incorporated, for example 3′-deoxyadenosine (cordycepin). The chain-terminating nucleoside blocks the addition of a 3′-terminal oligo(U) sequence to the mRNA containing the histone stem-loop. When the 3′-terminal oligo(U) sequence cannot be added, degradation of the mRNA is retarded. The mRNA then remains available to the translational machinery for a longer time, resulting in higher levels of protein synthesis.

The benefit of the Jan. 4, 2012 filing date of U.S. provisional patent application Ser. No. 61/583,043; and of the Dec. 7, 2012 filing date of U.S. provisional application Ser. No. 61/734,557 are claimed under 35 U.S.C. §119(e) in the United States, and are claimed under applicable treaties and conventions in all countries. The complete disclosures of both priority applications are hereby incorporated by reference in their entirety.

This invention was made with government support under grant numbers R01GM20818 and R01GM29832 awarded by the National Institute of General Medical Sciences of the National Institutes of Health. The government has certain rights in the invention.

### TECHNICAL FIELD

This invention pertains to stabilizing RNA molecules by incorporating chain-terminating nucleosides at the 3′ terminus, the use of these modified RNA molecules in peptide and protein synthesis, the use of these modified RNA molecules to promote translation, and other uses.

### BACKGROUND ART

Ribonucleic acid (RNA) is a single-stranded, linear polymer of ribonucleotides. Each ribonucleotide unit contains a nitrogenous base, a ribose sugar, and a phosphate group. There are several types of RNA molecules. Messenger RNA (mRNA) molecules are those whose nucleotide sequence determines the amino acid sequence of proteins. In eukaryotes, the 3′-ends of most mRNAs are polyadenylated; a so-called “poly(A) tail” is added to the 3′-end to promote translation and inhibit degradation of the mRNA by the exosome and other exonucleases. Polyadenylation also plays a role in transcription termination, export of mRNA from the nucleus to the cytosol, and translation. Polyadenylation regulates intracellular molecular activities, including RNA stability and translational efficiency.

The ability to synthesize RNA molecules in vitro with enhanced stability in cell culture, in vitro, or in vivo is useful because it allows one to prepare RNA molecules that can function more efficiently in a variety of biological applications. Such applications include both research applications and commercial production of polypeptides, e.g., producing in a cell-free translation system polypeptides containing an “unnatural” amino acid at a specific site, or producing in cultured cells polypeptides that require post-translational modification for activity or stability. mRNAs with enhanced stability will result in greater production of protein, whether for cultured cells, in vivo, or in vitro.

Stabilization of a specific mRNA in eukaryotic cells is of both research and commercial interest because the protein encoded by the mRNA can then be produced in larger quantities, due to a longer exposure of the mRNA to translational machinery. Enhanced production of proteins has many commercial and therapeutic applications. One application of particular interest is the production of cancer antigens for the purpose of immunizing patients against their own tumors. Cancer immunotherapy is an emerging therapy. Several drugs to enhance cancer immunotherapy are currently approved or in clinical trials. One approach to cancer immunotherapy is to introduce mRNAs encoding cancer antigens into dendritic cells, which are a type of antigen-presenting immune cells. See, e.g., Kuhn et al., 2011, Determinants of intracellular RNA pharmacokinetics: Implications for RNA-based immunotherapeutics. RNA Biol. 8, 35-43. Introducing genetic information through RNA rather than DNA allows transient expression of antigens, with essentially no possibility of the long-term complications that can result from the integration of exogenous DNA into the patient's chromosomes.

mRNA can be stabilized by incorporating a modified 7-methylguanosine-derived cap that cannot be cleaved by the intracellular pyrophosphatases that are part of the normal mRNA degradation machinery, such as Dcp2. An mRNA with an “uncleavable cap” is more stable within cells. See, e.g., Grudzien-Nogalska et al., 2007, Phosphorothioate cap analogs stabilize mRNA and increase translational efficiency in mammalian cells. RNA 13, 1745-1755; and Su et al., 2011, Translation, stability, and resistance to decapping of mRNAs containing caps substituted in the triphosphate chain with BH₃, Se, and NH. RNA 17, 978-988. Such modified-cap mRNAs have produced a more robust immunological response in animal models. See, e.g., Kuhn et al., 2010, Phosphorothioate cap analogs increase stability and translational efficiency of RNA vaccines in immature dendritic cells and induce superior immune responses in vivo. Gene Ther. 17, 961-971. See also U.S. Pat. Nos. 7,074,596 and 8,153,773

Increasing the length of the poly(A) tail and introducing stability elements from β-globin into the 3′-untranslated region has been reported to stabilize mRNA, as well as to increase the ability of dendritic cells to stimulate T-cells. See Holtkamp et al., 2006, Modification of antigen-encoding RNA increases stability, translational efficacy, and T-cell stimulatory capacity of dendritic cells. Blood 108, 4009-4017. Introducing “uncleavable caps” has been reported to produce mRNA having even greater stability, and also to stimulate a greater T-cell response in an animal model. See Kuhn et al., 2010.

Cordycepin, 3′-deoxyadenosine, is a chain terminator that both stops mRNA elongation by RNA polymerase and prevents polyadenylation by poly(A) polymerase after cordycepin has been incorporated at the 3′ terminus of an mRNA molecule. See Beach L R, Ross J. 1978. Cordycepin, an inhibitor of newly synthesized globin messenger RNA. J Biol Chem 253: 2628-2632.

United States patent application publication no. 2008/020706 discloses a method of mRNA production for use in transfection that involves in vitro transcription of PCR-generated templates with specially designed primers, followed by poly(A) addition, to produce a construct containing sequences in the 3′ and 5′ untranslated regions (“UTR”), a 5′ cap or Internal Ribosome Entry Site (IRES), the gene to be expressed, and a poly(A) tail, typically 50-200 bases in length. It was reported that RNA transfection can be effective in cells that are difficult to transfect efficiently with DNA constructs. It was reported that protein expression could be increased either by extending the length of the poly(A) tail, or by replacing ATP with the modified ATP analog cordycepin or 8-azaadenosine. It was speculated that poly(A) extension or the use of an ATP analog may enhance protein expression by better protecting the mRNA from 3′-exonuclease degradation. See also Rabinovich et al., 2006, Synthetic messenger RNA as a tool for gene therapy, Hum. Gene Ther. 17: 1027-1035.

The mRNAs that encode replicative histones (those that are involved in DNA synthesis) are unusual. Histone mRNAs have significant differences from most other mRNA molecules found in eukaryotes. Histone mRNAs are transcribed from genes that do not contain introns, and they do not contain the usual 3′-terminal poly(A) tail. Instead, these mRNAs have a unique ˜25 or ˜26 nucleotide 3′-terminal stem-loop (SL) secondary structure, located within the 3′-UTR at the 3′ end, that both stabilizes the mRNA against intracellular degradation and promotes translational efficiency. By contrast, poly(A) mRNAs do not contain an SL but rather contain a 3′-terminal poly(A) tract of ˜25-300 (or longer) nucleotides. See Marzluff et al., 2008, Metabolism and regulation of canonical histone mRNAs: life without a poly(A) tail. Nat. Rev. Genet. 9, 843-854. Histone mRNAs are stabilized during DNA synthesis and are degraded once DNA synthesis ceases. An early step in histone mRNA degradation is the addition of uridyl residues to the 3′-terminus, forming an oligo(U) tail, which in turn recruits mRNA degradation enzymes. See Mullen & Marzluff, 2008, Degradation of histone mRNA requires oligouridylation followed by decapping and simultaneous degradation of the mRNA both 5′ to 3′ and 3′ to 5′. Genes Dev. 22, 50-65. According to the Mullen and Marzluff model, histone mRNA is circularized during active translation. See Cakmakci N G, Lerner R S, Wagner E J, Zheng L, Marzluff W F. 2008. SLIP1, a factor required for activation of histone mRNA translation by the stem-loop binding protein. Mol Cell Biol 28: 1182-1194. When DNA synthesis stops, the regulator of nonsense transcripts 1 protein (Upf1) binds to the 3′ end of histone mRNA, followed by oligouridylation, and then degradation. Histone mRNA is an ancient and early-evolved type of mRNA molecule in eukaryotes. Eukaryotes have developed a highly-conserved machinery to degrade SL-containing mRNAs, one that differs substantially from the machinery that is used for degrading the more common, polyadenylated mRNAs.

The SL is recognized by a stem-loop binding protein (SLBP) that is essential for histone pre-mRNA processing, as well as for translation and regulated stability. See Gallie D R, Lewis N J, Marzluff W F. 1996. The histone 3′-terminal stem-loop is necessary for translation in Chinese hamster ovary cells. Nucleic Acids Res 24: 1954-1962; Wang Z-F, Whitfield M L, Ingledue III T C, Dominski Z, Marzluff W F. 1996. The protein that binds the 3′ end of histone mRNA: A novel RNA-binding protein required for histone pre-mRNA processing. Genes Dev 10: 3028-3040; Sanchez R, Marzluff W F. 2002. The stem-loop binding protein is required for efficient translation of histone mRNA in vivo and in vitro. Mol Cell Biol 22: 7093-7104.

By contrast, the poly(A) tract is recognized by entirely different binding proteins, the nuclear and cytoplasmic poly(A)-binding proteins (PABPs). PABPs are involved in pre-mRNA processing, translation, and stability.

The SL-containing histone mRNAs are transcribed from genes that do not contain introns and hence do not undergo a process of precursor maturation by exon splicing. By contrast, poly(A)-containing mRNAs are transcribed from genes containing introns, and in eukaryotic cells (including human cells), the great majority of these poly(A)-containing mRNAs (>99% of all mRNAs) must undergo splicing for maturation and export from the nucleus.

The mechanisms for degrading SL-containing and poly(A)-containing mRNAs are quite different. The stability of SL-containing mRNAs changes dramatically during the cell cycle; whereas most poly(A)-containing mRNAs are not regulated as a function of the cell cycle, and those that are sensitive to the phase of the cell cycle are instead regulated by different mechanisms. Specifically, SL-containing mRNAs are stable while DNA is being synthesized (during S phase), and they become unstable when DNA synthesis stops (either during other phases of the cell cycle, or when DNA synthesis is blocked during S phase by drugs such as hydroxyurea or cytosine arabinoside). See Kaygun H, Marzluff W F. 2005. Regulated degradation of replication-dependent histone mRNAs requires both ATR and Upf1. Nat Struct Mol Biol 12: 794-800. Once DNA synthesis stops, Upf1 binds to SLBP, and the resulting complex in turn recruits a terminal uridyltransferase (TUTase) that catalyzes 3′-oligouridylation. The oligo(U) tract forms a binding site for the Lsm1-7 heptamer, which then recruits the machinery for decapping and bidirectional degradation of histone mRNA by exoribonucleases (Mullen & Marzluff, 2008).

By contrast, poly(A)-containing mRNAs undergo progressive shortening by deadenylation of the poly(A) tract until they reach a length where PABP is unable to bind (less than ˜25 nt). The residual oligo(A) tract forms a binding site for the Lsm1-7 heptamer. Deadenylation leads to decapping by the Dcp1-Dcp2 complex at the 5′ end, followed by 5′-to-3′ exonucleolytic digestion of the RNA by Xrn1. Alternatively, the mRNA can be degraded from the 3′ end by the exosome. See Chen C Y, Shyu A B. 2011. Mechanisms of deadenylation-dependent decay. Wiley Interdiscip Rev RNA 2: 167-183.

Note particularly that the degradation of SL-containing mRNAs requires the addition of nucleotide residues (specifically, U residues), whereas the degradation of poly(A)-containing mRNAs requires the removal of nucleotide residues (specifically, A residues). The two processes are quite distinct.

The processing and stability of microRNAs (miRNAs) are also regulated via oligouridylation-dependent pathways. For regulation of processing, see Hagan, J P, Piskounova, E, Gregory, R I. 2009. Lin28 recruits the TUTase Zcchc11 to inhibit let-7 maturation in mouse embryonic stem cells. Nat Struct Mol Biol 16: 1021-1025; and Lehrbach et al., 2009, LIN-28 and the poly(U) polymerase PUP-2 regulate let-7 microRNA processing in Caenorhabditis elegans. Nat Struct Mol Biol 16, 1016-1020.

For regulation of miRNA stability via an oligouridylation-dependent pathway, see Li J, Yang Z, Yu B, Liu J, & Chen X. 2005. Methylation protects miRNAs and siRNAs from a 3′-end uridylation activity in Arabidopsis. Curr Biol 15: 1501-1507. The authors concluded that that 3′-end methylation is a common step in miRNA and siRNA metabolism, and that such methylation may protect the 3′ ends of small RNAs from uridylation activity. It was speculated that perhaps the same enzyme targets unmethylated small RNAs for uridylation as well, subsequently leading to the degradation of the small RNAs. It was also suggested that 3′-to-5′ exonuclease activity appears to be counteracted by 3′ methylation. It was speculated that the methylation of miRNAs and siRNAs by the enzyme HENT may also prevent RNA-dependent RNA polymerases from using the small RNAs as primers.

Ramachandran V, & Chen X. 2008. Degradation of microRNAs by a family of exoribonucleases in Arabidopsis. Science 321: 1490-1492 described the exonucleases responsible in plant cells, and concluded that one of these, SDN1, acts specifically on single-stranded miRNAs in vitro, and that this enzyme is sensitive to a 2′-O-methyl modification on the 3′ terminal ribose of miRNAs.

Ameres S L, Horwich M D, Hung J-H, Xu J, Ghildiyal M, Weng Z, & Zamore P D. 2010. Target RNA-directed trimming and tailing of small silencing RNAs. Science 328: 1534-1539 described modifications to miRNA in animal cells that affected the trimming and tailing of miRNAs when the miRNAs were bound to Argonaute 1 or 2 proteins (catalytic components of the RNA-induced silencing complex) in Drosophila. It was reported that 2′-O-methylation prevented trimming and tailing, and that a 3′-terminal, 3′-deoxy modification also inhibited target-directed effects. The authors suggested that methylation of small RNAs by the enzyme Hen1 may make them resistant to small RNA modifying and trimming enzymes.

See generally United States patent application publication no. 2009/0093433. See also US patent application publication no. 2011/0086904, U.S. Pat. No. 5,756,264, U.S. Pat. No. 5,807,707, international patent application WO 2008/148575, and international patent application WO 2007/065602.

### DISCLOSURE OF THE INVENTION

We have discovered a method to stabilize histone stem-loop-containing mRNA by the addition of a chain-terminating nucleoside. The mRNA contains a 3′ histone stem-loop (SL) sequence within the 3′ UTR. At the 3′ end of the 3′ UTR a chain-terminating nucleoside is incorporated, for example 3′-deoxyadenosine (cordycepin). The chain-terminating nucleoside blocks the addition of a 3′-terminal oligo(U) tract to an mRNA containing the histone stem-loop. When the 3′-terminal oligo(U) tract cannot be added, degradation of the mRNA is retarded. The mRNA is thereby stabilized, and more protein can then be synthesized as the mRNA is available to the translational machinery for a longer time.

A preferred chain-terminating nucleoside is 3′-deoxyadenosine (cordycepin). Other chain-terminating nucleosides may also be used, including for example 3′-deoxyuridine, 3′-deoxycytosine, 3′-deoxyguanosine, or 3′-deoxythymine. Other modifications to the 3′ end of the RNA that prevent or inhibit oligo(U) addition may also be used. Other examples include 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or a 2′-O-methylnucleoside. Likewise, an oligonucleotide that terminates in a 3′-deoxynucleoside or in a 2′,3′-dideoxynucleoside may also be used; as may 3′-O-methylnucleosides, 3′-O-ethylnucleosides, 3′-arabinosides, and other modified nucleosides. For example, there is at least one RNA polymerase, derived from bacteriophage T7, that will not add a nucleoside triphosphate to a terminal 2′-deoxyguanosine or 2′-O-methylguanosine. See Jemielity, J, Fowler, T, Zuberek, J, Stepinski, J, Lewdorowicz, M, Niedzwiecka, A, Stolarski, R, Darzynkiewicz, E, Rhoads, R E. 2003. Novel “anti-reverse” cap analogues with superior translational properties. RNA 9: 1108-1122.

Interestingly, we have observed that adding cordycepin or other modified nucleosides can also stabilize mRNAs that have an ordinary 3′-poly(A) tail. See FIG. 5. But in that case, unlike an SL-containing RNA, the stabilization also required a cap structure that is resistant to cleavage by the decapping enzyme Dcp2 (FIG. 5B and Table III). In RNAs that lacked a decapping-resistant cap structure, we observed only minimal stabilization of the 3′ sequences of poly(A) mRNA, and no stabilization of 5′ sequences following the 3′-addition of cordycepin (FIG. 5A and Table III). These observations further confirmed that poly(A)-containing RNA and SL-containing RNA are degraded by different mechanisms. Our observations suggest that a previously unreported mechanism is likely to be involved.

Stabilizing a protein-encoding mRNA leads to greater protein production in cells. The novel method can thus be used to increase the synthesis of specific proteins. A particularly promising example is the use of the novel method in cancer immunotherapy, to introduce mRNA that encodes cancer-specific antigens into dendritic cells. There are many additional applications of the novel method, which can be incorporated into any of the hundreds of biotechnology techniques that are based upon protein production. The novel method may be used to stabilize mRNA in the production of any physiological or non-physiological protein.

In prototype experiments we have successfully blocked the addition of a 3′-terminal oligo(U) tail in an mRNA by synthesizing an SL-containing mRNA that encoded luciferase and that had a cordycepin at the 3′-terminus. The cordycepin modification stabilized the mRNA relative to unmodified mRNA. More luciferase will be synthesized with the modified mRNA than with an mRNA lacking cordycepin, because the mRNA remains available to the translational machinery longer, and because addition of cordycepin does not affect the translational efficiency of the mRNA. See FIG. 4. Our observations suggested that the recruitment of mRNA degradation machinery was retarded by cordycepin-modified mRNA, likely because oligouridylation was prevented.

### BRIEF DESCRIPTION OF THE DRAWINGS

FIGS. 1A-1B illustrate the stabilizing effect of the chain-terminator cordycepin on ARCA-Luc-SL mRNA and BTH-Luc-SL mRNA in HeLa cells. FIG. 1A shows the loss of 5′ and 3′ sequences for ARCA-Luc-SL with (filled symbols) or without (open symbols) cordycepin modification. FIG. 1B shows the loss of 5′ and 3′ sequences for BTH-Luc-SL with (filled symbols) or without (open symbols) cordycepin modification.

FIGS. 2A-2B illustrate the absence of any stabilizing effect of cordycepin on ARCA-Luc-SL and BTH-Luc-SL that contained a 10-nt oligo(U) tract prior to nucleoporation. FIG. 2A shows the loss of 5′ and 3′ sequences for ARCA-Luc-SL-U₁₀ with (filled symbols) or without (open symbols) cordycepin modification. FIG. 2B shows the loss of 5′ and 3′ sequences for BTH-Luc-SL-U₁₀ with (filled symbols) or without (open symbols) cordycepin modification.

FIGS. 3A-3B illustrate the stabilizing effect of cordycepin on ARCA-Luc-SL and BTH-Luc-SL that were added with hydroxyurea (HU) treatment. FIG. 3A shows the destabilization of ARCA-Luc-SL with the addition of HU immediately after nucleoporation (open symbols), and the stabilization of ARCA-Luc-SL with cordycepin modification prior to the addition of HU treatment (filled symbols). FIG. 3B shows the destabilization of BTH-Luc-SL with the addition of HU immediately after nucleoporation (open symbols), and shows the stabilization of BTH-Luc-SL with cordycepin modification prior to the addition of HU treatment (filled symbols).

FIGS. 4A-4C illustrate the effect of cordycepin incorporation and the translational efficiency of cordycepin-modified mRNAs. FIG. 4A shows that 3′-terminal addition of cordycepin was over 95% effective in preventing further addition of ATP by poly(A) polymerase. FIG. 4B shows that addition of 3′-terminal cordycepin to either ARCA-Luc-SL or BTH-Luc-SL did not significantly alter their translational efficiencies in HeLa cells. The results shown in FIG. 4B are corrected for the amount of mRNA present; because there was more mRNA in the cordycepin samples at later times, there was greater total protein production. FIG. 4C shows that the addition of 3′-terminal cordycepin to either ARCA-Luc-SL-U₁₀ or BTH-Luc-SL-U₁₀ did not significantly alter their translational efficiencies in HeLa cells.

FIGS. 5A-5B illustrate the effect of cordycepin incorporation on polyadenylated mRNA and its rate of degradation when the mRNA contained an uncleavable cap. FIG. 5A shows that the loss of 5′ and 3′ sequences for ARCA-Luc-A₇₄ with (filled symbols) or without (open symbols) cordycepin modification showed no statistically significant difference. FIG. 5B shows that the loss of 5′ and 3′ sequences of BTH-Luc-A₇₄ was significantly slowed by cordycepin modification (filled symbols) compared to unmodified BTH-Luc-A74 (open symbols).

### MODES FOR CARRYING OUT THE INVENTION

### EXAMPLE 1

### Materials and Methods

Materials. All common reagents were of analytical grade. ARCA and BTH cap analogs were synthesized as previously described (Jemielity et al., 2003, Novel “anti-reverse” cap analogues with superior translational properties. RNA 9, 1108-1122; and Su et al., 2011).

In vitro synthesis of mRNA. pLuc-A₆₀ was constructed as previously described (Grudzien et al., 2006, Differential inhibition of mRNA degradation pathways by novel cap analogs. J Biol Chem 281, 1857-1867). pT7-Luc-SL and pT7-Luc-TL were constructed and linearized as previously described (Gallie et al., 1996, The histone 3′-terminal stem-loop is necessary for translation in Chinese hamster ovary cells. Nucleic Acids Res 24, 1954-1962. The linearized plasmids served as templates for in vitro synthesis of mRNAs as previously described (Su et al., 2011).

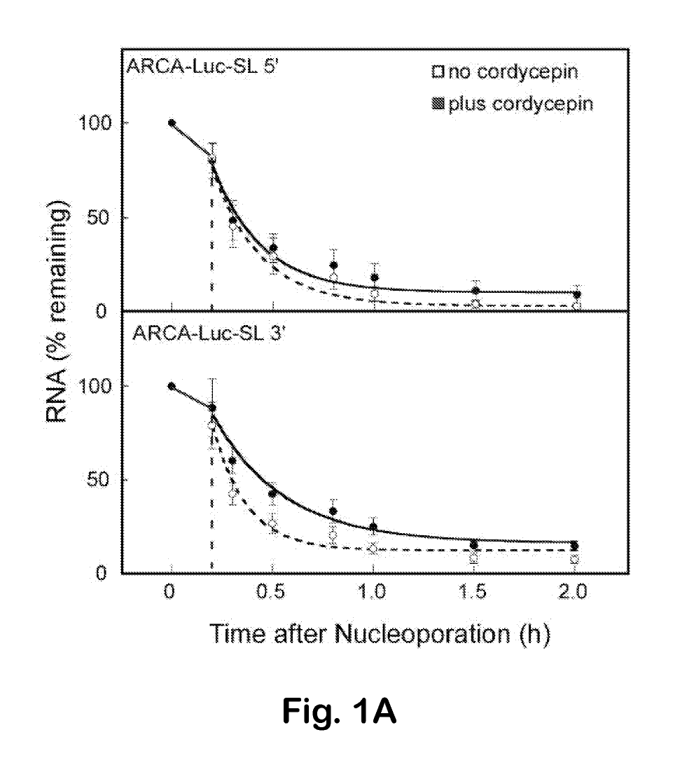

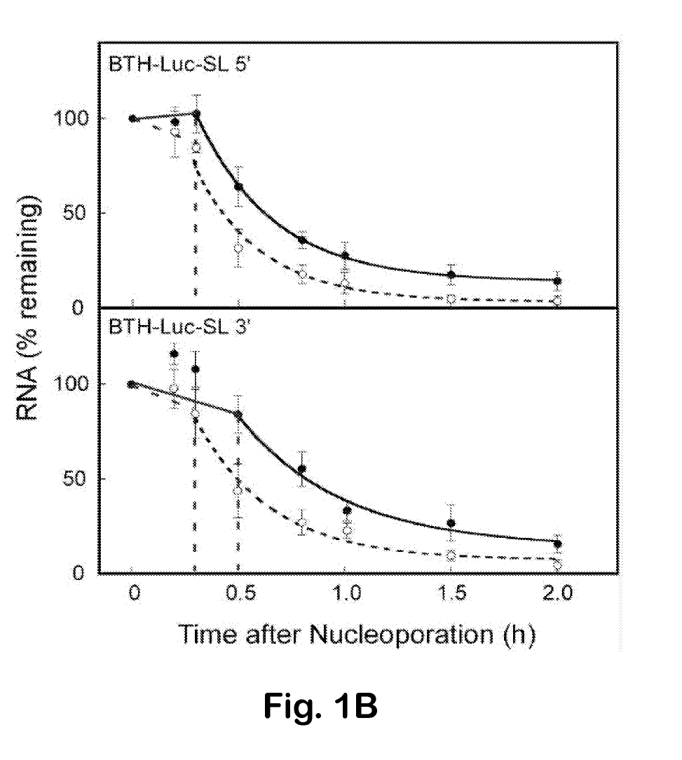

The SL sequence used in the DNA constructs to generate the mRNA was 5′ CAAAGGTCTTTTCAGAGCCAC 3′ (SEQ ID NO:7), reflecting the structure of the cytosolic histone mRNA that results from trimming three nucleotide residues from the histone mRNA after processing in the nucleus. See Mullen and Marzluff, 2008, and FIG. 1 in Su et al., 20133, mRNAs containing the histone 3′ stem-loop are degraded primarily by decapping mediated by oligouridylation of the 3′ end. RNA 19, 1-16. The sequences of other histone stem-loops are known in the art, and are given, for example, in Marzluff et al., 2008, Metabolism and regulation of canonical histone mRNAs: life without a poly(A) tail. Nat. Rev. Genet. 9, 843-854; and in Martin et al., 2012, Systematic reconstruction of RNA functional motifs with high-throughput microfluidics. Nature Methods. 9, 1192-1194. See also Table I.

TABLE I
Histone stem-loops sequences
from various species^{a}

| Species | Sequence |
|---|---|
| Homo sapiens | CCAAAGGCUCUUUUCAGAGCCACCCA |
| | (SEQ ID NO: 8) |
| Drosophila | CCAAAAGUCCUUUUCAGGGCUACCA |
| melanogaster | (SEQ ID NO: 9) |
| Caenorhabditis | CCAACGGCCCUCUUUAGGGCCACAAA |
| elegans | (SEQ ID NO: 10) |
| Dictyostelium | CCAAAGGUGUUUUUUAACACCACACA |
| discoideum | (SEQ ID NO: 11) |
| Trichomonas | UAAUUGGAUUUUUUCAAAUCCACCUG |
| vaginalis | (SEQ ID NO: 12) |
| Plasmodium | UUUGAGGCUUCCCUAGAAGCCAUUAC |
| berghei | (SEQ ID NO: 13) |
| Tetrahymena | GUUACAGGUAUAUUUAUCCCUACUAA |
| thermophila | (SEQ ID NO: 14) |
| Mus musculus | CCAACGGCUCUUUUCAGAGCCACCCA |
| | (SEQ ID NO: 15) |
| Volvox carteri | AACCCGGUGUUUUUUAACACCACCGC |
| | (SEQ ID NO: 16) |
| Chlamydomonas | AACUCGGUGUUUCUCAACACCACCUA |
| reinhardtii | (SEQ ID NO: 17) |

^{a}Data are from Marzluff et al., 2008, Nat. Rev. Genet. 9, 843-854; and Martin et al., 2012, Nature Methods 9, 1192-1194.

Cell culture and nucleoporation. HeLa cells were cultured as previously described (Su et al., 2011). Asynchronous cells were seeded 1 day prior to nucleoporation onto 150-mm dishes at a density such that cells would reach 70% confluency the next day. Cells for synchronization (1×10⁶) were seeded onto 150-mm dishes and synchronized by double-thymidine block (following the procedure of Jackman & O'Connor, 2001, Methods for synchronizing cells at specific stages of the cell cycle. Current Protocols in Cell Biology: John Wiley & Sons, Inc). Cells were released from double-thymidine block on the day of nucleoporation, detached from plates 3 h after release (middle of S-phase), and subjected to nucleoporation as described previously (Su et al., 2011).

Measurement of translational efficiency and mRNA decay in HeLa cells. To assay translational efficiency in cultured cells, aliquots of 0.5×10⁵ cells were shaken in 1.5-ml Eppendorf tubes at 37° C. for various times after nucleoporation. Total protein was extracted and luciferase activity was measured and normalized to the amount of luciferase mRNA delivered into the cell at time zero, as described previously (Grudzien-Nogalska et al., 2007). For measurement of mRNA stability over periods shorter than 1 h, cells were shaken in Eppendorf tubes as described above. For periods longer than 1 h, cells were plated onto 35-mm cell culture dishes and incubated at 37° C. in 5% CO₂. Total RNA was extracted as described previously (Grudzien-Nogalska et al., 2007). Luciferase mRNA was quantified by qRT-PCR using primers designed with the Beacon Designer tool (Bio-Rad). Sequences from the 5′-end of Luc mRNA were amplified with 5′-GGATGGAACCGCTGGAGAG-3′ (SEQ ID NO:1) and 5′-GCATACGACGATTCTGTGATTTG-3′ (SEQ ID NO:2). Sequences from the 3′-end of Luc mRNA were amplified with 5′-ATCGTGGATTACGTCGCCAGTCAA-3′ (SEQ ID NO:3) and 5′-TTTCCGCCCTTCTTGGCCTTTATG-3′ (SEQ ID NO:4). Human 18S rRNA levels were measured by the same method and in the same RNA samples with primers 5′-CGAGCCGCCTGGATACC-3′ (SEQ ID NO:5) and 5′-CAGTTCCGAAAACCAACAAAATAGA-3′ (SEQ ID NO:6). Amplification and detection were performed with the iCycler IQ real time PCR detection system in 25-μl reaction mixtures containing 5 μl of the transcription reaction mixture (50 ng of cDNA), 12.5 μl of IQ SYBRgreen Supermix, and 0.3 mM primers (Bio-Rad). The incubation conditions were 3 min at 95° C. for polymerase activation, followed by 40 cycles, of 15 s each at 95° C. and 1 min each at 60° C. Luciferase mRNA levels were calculated using the absolute standard curve method as described in the iCycler iQ™ Real-time PCR Detection System Instruction Manual (catalog number 170-8740). Luciferase mRNA was normalized for the amount of 18S rRNA in each sample, which is an indicator of total cellular RNA purified from each cell extract. KaleidaGraph (Synergy Software, Reading, Pa., version 3.06) was used for nonlinear least-squares fitting of decay data.

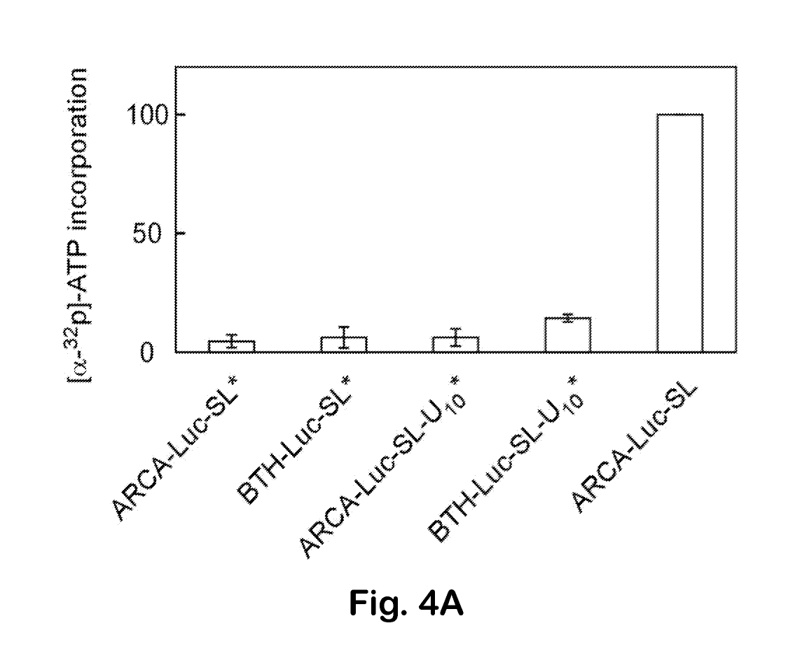

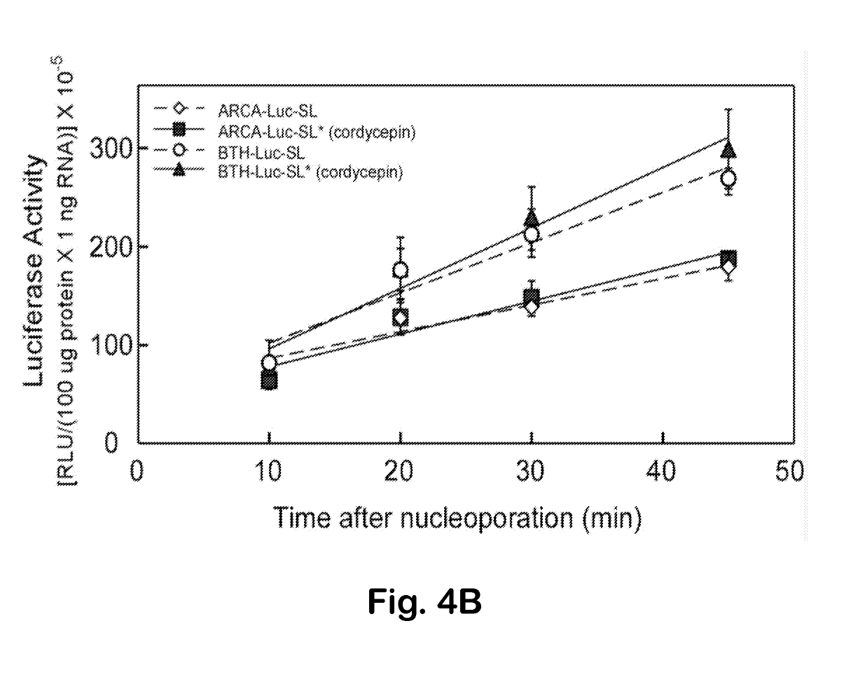

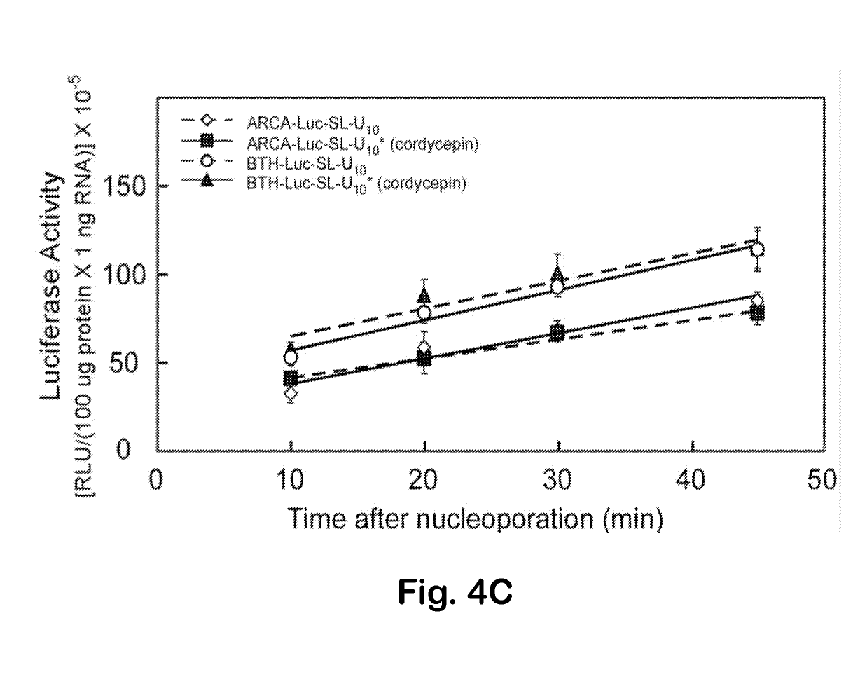

Cordycepin incorporation. Cordycepin to produce either ARCA-Luc-SL* or BTH-Luc-SL* was incorporated via a 100-μl reaction mixture that contained 0.2 μM ARCA-Luc-SL or BTH-Luc-SL mRNA, respectively, 1× poly(A) polymerase (PAP) reaction buffer (Affymetrix), 100 μM cordycepin 5′-triphosphate (Sigma), 1 U/μl of RNase Inhibitor (Applied Biosystems), and 2400 units of yeast PAP (Affymetrix). The reaction mixture was incubated at 37° C. for 1 h. To confirm the extent of cordycepin incorporation, 5 μl of the reaction mixture were removed and incubated with [α-³²P]ATP and fresh PAP for an additional 1 hr. at 37° C. RNA was separated from unincorporated nucleoside triphosphates with a NucAway spin column (Ambion), and the ³²P content of RNA was measured by Cerenkov radiation. These observations indicated that over 95% of the RNA had been successfully modified with cordycepin (FIG. 4A). The remainder of the PAP reaction mixture was extracted with phenol and chloroform. RNAs were precipitated by adding two volumes of Precipitation/Inactivation buffer (Ambion), kept at −20° C. for at least 1 h, and collected by centrifugation at 13,000×g at 4° C. for 20 min. RNA was resuspended in 30 μl of diethylpyrocarbonate (DEPC)-treated water. The RNA concentration was determined by UV absorbance at 260 nm, and RNA integrity was verified by electrophoresis on 1.2% agarose gels containing formaldehyde.

### EXAMPLES 2-5

Four mRNAs were synthesized in vitro by transcription of a plasmid via T7 RNA polymerase: ARCA-Luc-SL, ARCA-Luc-SL*, BTH-Luc-SL, and BTH-Luc-SL*. ARCA-Luc-SL contained: i) an “anti-reverse cap analog” (ARCA) at the 5′-end, ii) the coding region of firefly luciferase mRNA, and iii) the 3′-untranslated region of a histone mRNA at the 3′-end, including the SL.

Anti-reverse cap analogs (ARCAs) are described in U.S. Pat. No. 7,074,596. They can be used to prevent the incorporation of the cap dinucleotide in the reverse orientation during the T7 RNA polymerase reaction. Typical ARCAs are m₂^{7,3}′^{-O}GpppG (See Stepinski et al., 2001, Synthesis and properties of mRNAs containing the novel “anti-reverse” cap analogues 7-methyl(3′-O-methyl)GpppG and 7-methyl(3′-deoxy)GpppG. RNA 7, 1486-1495) and m₂^{7,2}′^{-O} GpppG (See Jemielity et al., 2003, Novel “anti-reverse” cap analogues with superior translational properties. RNA 9, 1108-1122). In cultured cells, mRNAs containing the natural cap (m⁷GpppG) do not differ substantially either in translational efficiency or in stability from mRNAs containing ARCAs, presumably because the 2′- and 3′-positions of the guanosine moiety in m⁷GpppG are not involved in cap recognition by the translational cap-binding protein eIF4E, or by the decapping pyrophosphatase Dcp2.

ARCA-Luc-SL* was the same as ARCA-Luc-SL, except that a 3′-terminal cordycepin residue was incorporated, as described above.

BTH-Luc-SL contained an alternative cap, m⁷Gpp_{BH3}pm⁷G, in which a β non-bridging oxygen atom was substituted with BH₃. The BTH cap analog (Borano Two-Headed) is described in U.S. Patent Application Publication No. 2011/0092574 and in Su et al., 2011. The BH₃ modification inhibits cleavage of pyrophosphate by Dcp2, and thus stabilizes mRNA in vivo by retarding 5′→3′ degradation.

BTH-Luc-SL* was the same as BTH-Luc-SL, except that a 3′-terminal cordycepin residue was incorporated, as described above.

ARCA-Luc-SL and BTH-Luc-SL were synthesized by in vitro transcription of the plasmid pT7-Luc-SL, which contains the firefly luciferase coding region under control of the T7 promoter, and a wild-type histone mRNA 3′-untranslated region containing the SL at the 3′-end (Gallie et al., 1996, The histone 3′-terminal stem-loop is necessary for translation in Chinese hamster ovary cells. Nucleic Acids Res. 24, 1954-1962). The plasmid was cut with restriction enzyme AflII at a site immediately downstream of the SL. ARCA-Luc-SL and BTH-Luc-SL were synthesized by T7 polymerase in the presence of ARCA and BTH, respectively. The procedures for synthesis and purification of mRNAs were as otherwise described in Su et al., 2011.

Four additional mRNAs were synthesized to study the effect of pre-uridylating the mRNAs. We synthesized pre-uridylated reporter mRNAs by inserting 10 T residues in the DNA template after the sequence for Luc-SL, resulting in an mRNA that contained 10 U residues located 3′ to the SL. Both ARCA-Luc-SL-U₁₀ and BTH-Luc-SL-U₁₀ were synthesized as otherwise described above. Each was modified with cordycepin to produce ARCA-Luc-SL-U₁₀* and BTH-Luc-SL-U₁₀*, respectively, as otherwise described above.

### EXAMPLES 6-9

Four additional mRNAs were synthesized to study the effect of 3′-terminal cordycepin on the stability of polyadenylated mRNAs: ARCA-Luc-A₇₄, ARCA-Luc-A₇₄*, BTH-Luc-A₇₄, and BTH-Luc-A₇₄*. The methods were otherwise the same as those described above for the various SL-containing mRNAs, except that the plasmid used as a template for in vitro transcription by T7 RNA polymerase was pluc-A₇₄, rather than pT7-Luc-SL. See Grudzien-Nogalska et al., 2013, Synthetic mRNAs with superior translation and stability properties, in Meth. Mol. Biol. Synthetic Messenger RNA and Cell Metabolism Modulation Methods and Protocols Series: Methods in Molecular Biology, Vol. 969, Rabinovich, Peter M. (Ed.), pp. 55-72. The mRNAs transcribed from pluc-A₇₄ contained the coding region for firefly luciferase followed by a poly(A) tract of 74 nucleotide residues, with no heterologous (non-A) nucleotide residues downstream from the poly (A) tract.

### EXAMPLES 10-17

The various mRNAs were introduced into HeLa cells by nucleoporation. HeLa cells were synchronized by double thymidine block, and the various mRNAs were introduced at S phase by nucleoporation. Cells were lysed at the indicated times, and Luc-SL mRNA was measured by quantitative real time PCR using primer sets that amplified sequences at either the 5′-end or the 3′-end of Luc-SL mRNA. Data were plotted as a percentage of the luciferase mRNA present immediately after nucleoporation. The decay patterns for ARCA-Luc-SL and BTH-Luc-SL were both biphasic, with a lag phase followed by a rapid-decay phase (separated by the vertical dashed lines in FIG. 1). Sequences from the 5′-end of ARCA-Luc-SL* (FIG. 1A, filled symbols) were more stable than the same 5′-sequences from ARCA-Luc-SL (FIG. 1A, open symbols) at later times. This effect was much more pronounced when sequences at the 3′-end were monitored, suggesting that oligouridylation affected the 3′→5′ pathway more than the 5′→3′ pathway. BTH-Luc-SL* was also more stable than BTH-Luc-SL (FIG. 1B). Again, this effect was more pronounced at later times, and affected 3′ sequences more than 5′ sequences. For instance, the t_{1/2} for the rapid-decay phase increased from 21.5±1.5 to 39.9±5.5 min for 3′ sequences, but only from 30.2±4.4 to 33.3±8.0 for 5′ sequences (See Table II, lines 7 and 9). Our observation that cordycepin modification increased the stability of BTH-Luc-SL is further evidence that oligouridylation primarily affects the 3′→5′ pathway.

TABLE II
Decay of Luc-SL mRNAs with modified 3′ termini as a function of cell treatment

| | | | mRNA decay (min)^{b} | | | |
|---|---|---|---|---|---|---|
| | | | 5′ | | 3′ | |
| No. | mRNA^{a} | Treatment | lag | t_{1/2} | lag | t_{1/2} |
| 1 | ARCA-Luc-SL | | 7.8 ± 4.1 | 20.0 ± 3.1 | 7.5 ± 4.8 | 18.5 ± 1.0 |
| 2 | ARCA-Luc-SL | HU | 0 | 12.8 ± 1.9 | 0 | 12.5 ± 0.8 |
| 3 | ARCA-Luc-SL* | | 10.0 ± 2.8 | 24.7 ± 9.3 | 8.0 ± 4.8 | 22.7 ± 6.3 |
| 4 | ARCA-Luc-SL* | HU | 10.0 ± 0 | 17.0 ± 0 | 20.0 ± 0 | 19.8 ± 2.0 |
| 5 | ARCA-Luc-SL-U₁₀ | | 0 | 18.5 ± 1.9 | 0 | 20.6 ± 0 |
| 6 | ARCA-Luc-SL-U₁₀* | | 0 | 14.6 ± 1.0 | 0 | 20.5 ± 1.0 |
| 7 | BTH-Luc-SL | | 38.4 ± 7.0 | 30.2 ± 4.4 | 30.0 ± 0.5 | 21.5 ± 1.5 |
| 8 | BTH-Luc-SL | HU | 23.5 ± 5.0 | 27.1 ± 7.5 | 10.0 ± 5.0 | 13.3 ± 5.9 |
| 9 | BTH-Luc-SL* | | 27.5 ± 5.0 | 33.3 ± 8.0 | 31.3 ± 10.3 | 39.9 ± 5.5 |
| 10 | BTH-Luc-SL* | HU | 25.0 ± 7.1 | 29.3 ± 10.3 | 30.0 ± 0 | 35.9 ± 2.4 |
| 11 | BTH-Luc-SL-U₁₀ | | 20.0 ± 5.0 | 18.1 ± 0 | 10.0 ± 5.0 | 21.4 ± 0.5 |
| 12 | BTH-Luc-SL-U₁₀* | | 20.0 ± 5.0 | 20.9 ± 0 | 10.0 ± 5.0 | 19.7 ± 1.0 |

^{a}mRNAs followed by an asterisk (*) were modified at the 3′ end with cordycepin before nucleoporation.
^{b}t_{1/2} was calculated from 2 to 6 individual experiments. If there was a lag phase, the t_{1/2} was calculated for the post-lag period.

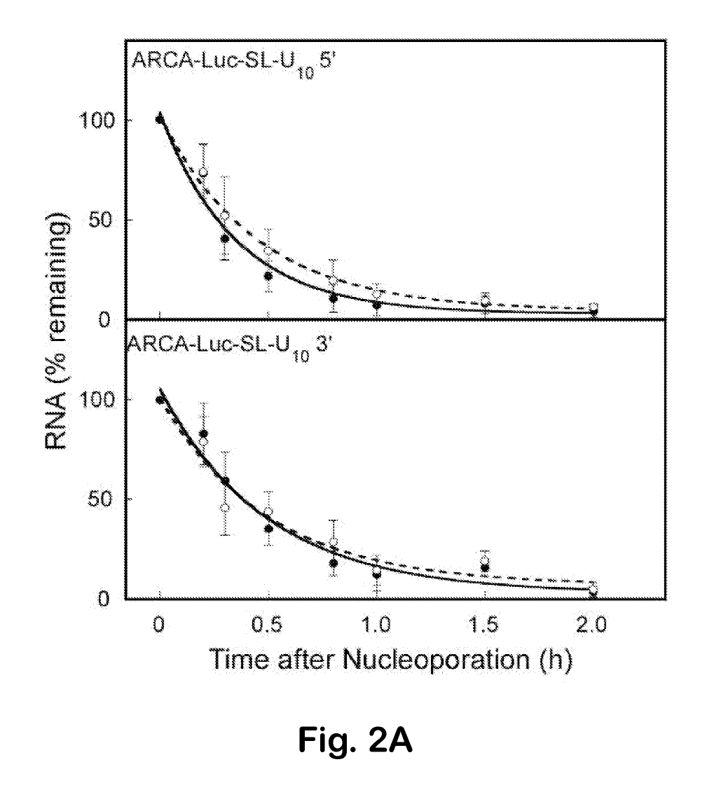

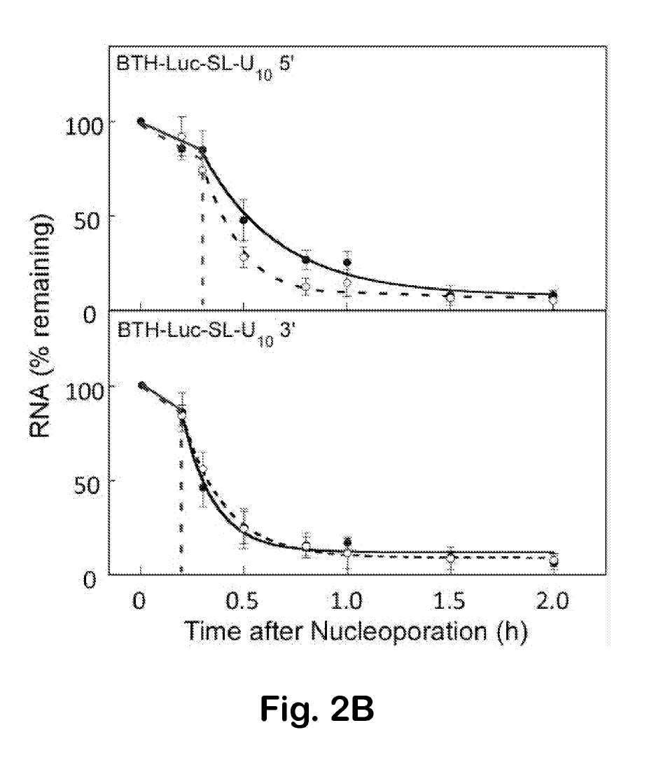

To rule out the possibility that the cordycepin modification might have interfered with some other step, a step that does not involve oligouridylation, a pre-uridylated reporter mRNA was synthesized by inserting 10 A residues in the DNA template after the sequence for Luc-SL, resulting in an mRNA that contained 10 U residues located 3′ to the SL. Both ARCA-Luc-SL-U₁₀ and BTH-Luc-SL-U₁₀ were synthesized, and each was modified with cordycepin to produce ARCA-Luc-SL-U₁₀* and BTH-Luc-SL-U₁₀*, respectively. The rapid-decay phase of ARCA-Luc-SL-U₁₀ was similar to that of ARCA-Luc-SL, but the lag phase did not occur for ARCA-Luc-SL-U₁₀, indicating that degradation of pre-uridylated mRNA begins immediately and suggesting that the oligo(U) tail efficiently recruits the degradation machinery (FIG. 2A versus 1A, open symbols; Table II, lines 1 and 5). Importantly, cordycepin did not diminish the degradation rate of ARCA-Luc-SL-U₁₀ (FIG. 2A, filled versus open symbols, and Table II, lines 5 and 6). For BTH-Luc-SL-U₁₀, a shorter lag phase was observed as compared to BTH-Luc-SL (FIG. 2B versus 1B, open symbols, and Table II, lines 7 and 11). Since 5′→3′ degradation was blocked for this mRNA, this observation was interpreted as showing that pre-uridylation accelerated the rate of 3′→5′ degradation. Cordycepin incorporation did not affect the rate at which either 5′ or 3′ sequences were lost from BTH-Luc-SL-U₁₀ mRNAs (FIG. 2B and Table II, lines 11 and 12). These results indicated that oligouridylation per se accelerated the rate of decay; and that our observations did not result from hypothetically blocking some other process by the cordycepin modification.

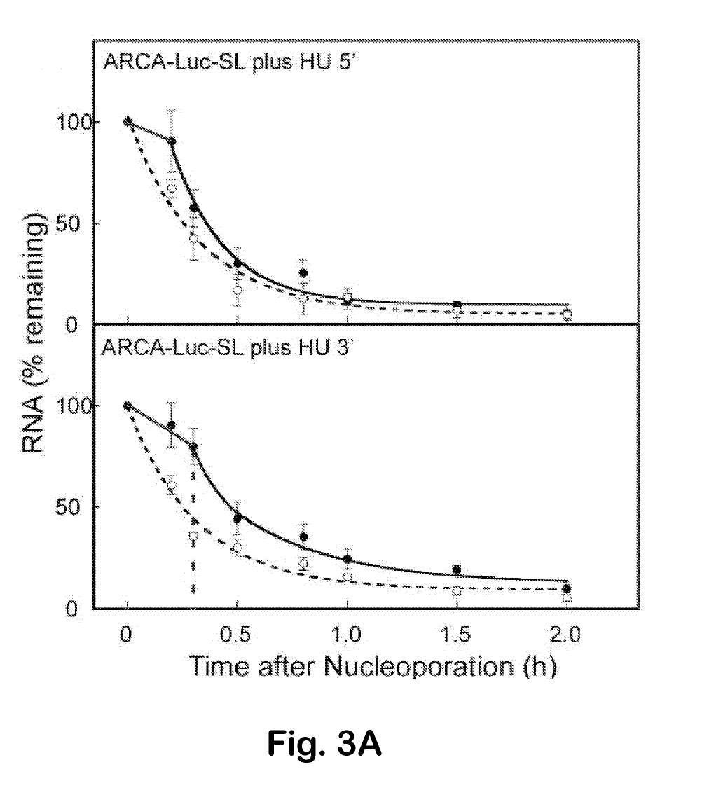

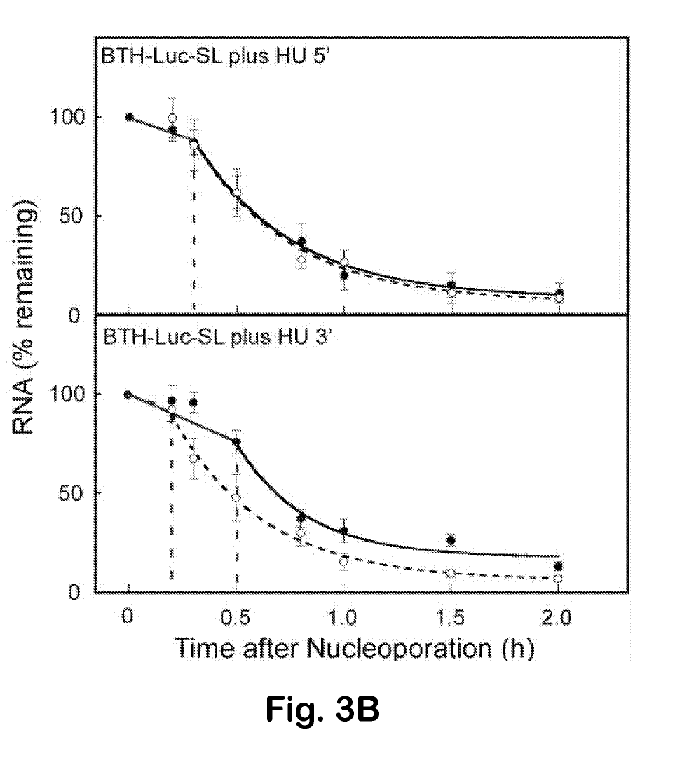

Cordycepin-modified, pre-uridylated mRNA was expected to be destabilized by HU treatment. However, we observed the opposite result. For BTH-Luc-SL*, HU treatment had no effect on the rate of loss of either 5′ or 3′ sequences during either the lag phase or the rapid-decay phase (FIG. 3B versus 1B, filled symbols, and Table II, lines 9 and 10). This observation was also true for ARCA-Luc-SL* (FIG. 3A versus 1A, filled symbols, and Table II, lines 3 and 4). In fact, the lag phase for decay of 3′ sequences was actually lengthened by HU treatment. These results indicated that the destabilization of Luc-SL mRNAs by HU treatment resulted from a pathway that involves oligouridylation, because inhibition of oligouridylation by cordycepin blocked the effect of HU on LUC-SL mRNA degradation, while oligouridylating the mRNA before transfection resulted in rapid degradation even without HU treatment.

### EXAMPLES 18-21

A series of experiments confirmed that the 3′-terminal cordycepin did not inhibit the ability of mRNA to direct protein synthesis. Translational efficiency in HeLa cells of ARCA-Luc-SL was compared to that of ARCA-Luc-SL*; parallel observations were also made for BTH-Luc-SL versus BTH-Luc-SL*. First, we demonstrated that incorporation of cordycepin blocked further addition of poly(A) by yeast poly(A) polymerase (FIG. 4A). Four types of mRNAs were modified by incubation of cordycepin 5′-triphosphate and yeast poly(A) polymerase (PAP) (as described in Su et al., 2013, with ARCA-Luc-SL*, BTH-Luc-SL*, ARCA-Luc-SL-U₁₀*, and BTH-Luc-SL-U₁₀*, where an asterisk (*) indicates the presence of 3′-terminal cordycepin. To determine the degree of cordycepin incorporation, a second reaction was carried out on aliquots of the original reaction mixtures with fresh PAP and [α-³²P]ATP. Incorporation of ³²P into the modified mRNAs was less than 5% of that of unmodified ARCA-Luc-SL (FIG. 4A).

We compared the ability of ARCA-Luc-SL and ARCA-Luc-SL* mRNA to produce luciferase after incorporation into HeLa cells (FIG. 4B). We had previously shown that mRNA capped with BTH has ˜2-fold higher translation efficiency (rate of protein production per unit of mRNA) as compared to the same mRNA capped with ARCA (Su et al., 2011). This result is replicated in FIG. 4B, which depicts results for ARCA-Luc-SL and BTH-Luc-SL synthesized in vitro and introduced into HeLa cells by nucleoporation, as otherwise described in Su et al., 2011. Cells were collected and lysed, and luciferase activity was measured in the lysates at the indicated times following nucleoporation. Data was normalized for the protein concentration and amount of luciferase mRNA present in each lysate. Consistent with the report of Su et al., 2011, the presence of the BTH cap increased translational efficiency by ˜2-fold as compared to that seen with the ARCA cap. FIG. 4B also depicts results for ARCA-Luc-SL* and BTH-Luc-SL*, each of which contained a 3′-terminal cordycepin residue. The translational efficiency of BTH-Luc-SL* was ˜2-fold higher than that of ARCA-Luc-SL* (FIG. 4B). Thus cordycepin modification was found to have no effect on the translational efficiency of either ARCA-Luc-SL or BTH-Luc-SL (FIG. 4B).

The observed translational efficiencies for ARCA-Luc-SL-U₁₀ and BTH-Luc-SL-U₁₀ were about half those of their unmodified counterparts (FIG. 4C). However, incorporation of cordycepin did not alter translational efficiencies (FIG. 4C). The diminished translation of pre-uridylated mRNAs may be due to interference in the SL-SLBP interaction by proteins that bind to oligo(U), such as Lsm1-7, which are also known to recruit inhibitors of translation (Coller & Parker, 2005, General translational repression by activators of mRNA decapping. Cell 122, 875-886).

### EXAMPLES 22-29

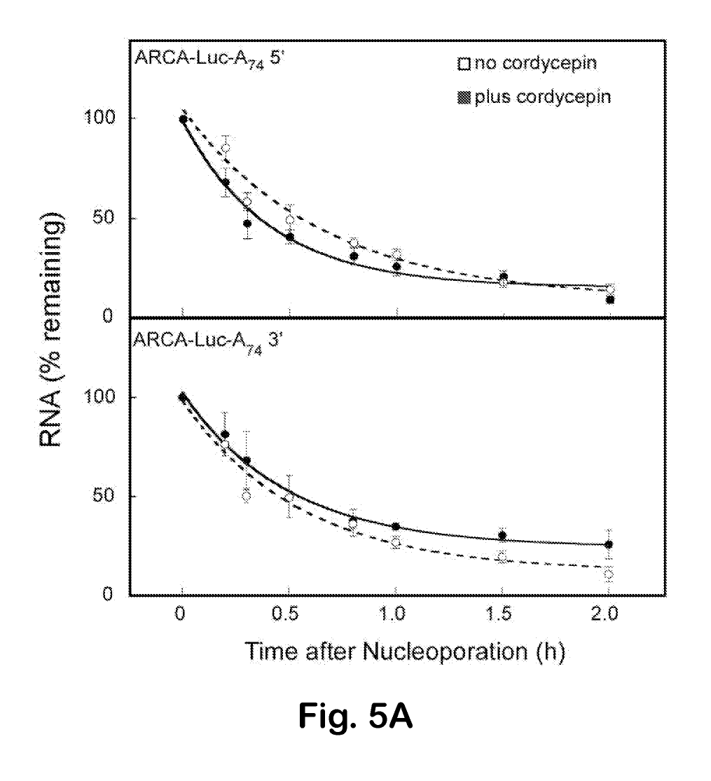

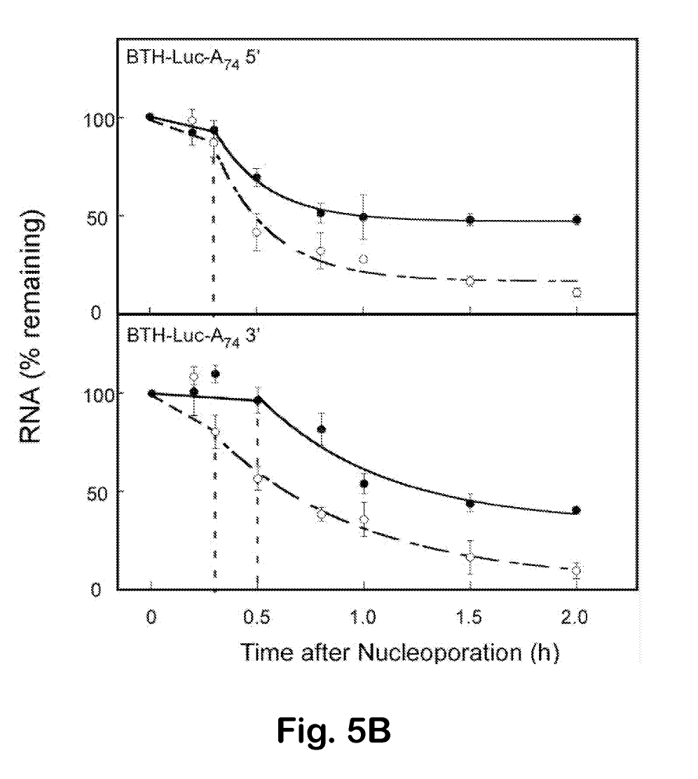

We also tested the effect of 3′-terminal cordycepin on the stability of polyadenylated mRNAs (FIGS. 5A-5B). There was no significant difference in the stability of the 5′-terminal sequences of ARCA-Luc-A₇₄ as compared to ARCA-Luc-A₇₄* mRNA (FIG. 5A, upper graph), nor was there a significant difference in the stability of their 3′-terminal sequences (FIG. 5A, lower graph). However, for BTH-Luc-A₇₄ mRNA, the cordycepin modification stabilized the 5′-terminal sequences (FIG. 5B, upper graph, filled versus open symbols), and the cordycepin stabilization of the 3′-terminal sequences was even greater (FIG. 5B, lower graph, filled versus open symbols). Thus, for both SL- and poly(A)-containing mRNAs, combining the 5′-modification (uncleavable cap) and the 3′-modification (chain-terminating nucleoside) resulted in an mRNA with greater stability than either the 5′-modification or the 3′-modification alone. The underlying mechanism is not known.

The effects of cordycepin on the amount of mRNA remaining at the endpoint of each of the experiments are summarized in Table III. The results demonstrated that there was a dramatic increase in the retention of both the 5′-terminal and 3′-terminal sequences of BTH-Luc-A₇₄ when cordycepin was added, but an increase only in the 3′-terminal sequences of ARCA-Luc-A₇₄. For both ARCA-Luc-SL and BTH-Luc-SL, cordycepin increased the abundance of both 5′- and 3′-terminal sequences. In the case of 3′-terminal sequences in BTH-Luc-SL, the increase was as much as 17-fold. Importantly, the stabilization of Luc-SL by cordycepin did not require the presence of an uncleavable cap, although the presence of an uncleavable cap did further enhance mRNA stability.

TABLE III
Messenger RNA remaining at end of experiment^{a}

| | 5′-sequences | | 3′-sequences | |
|---|---|---|---|---|
| | No | Plus | No | Plus |
| mRNA | cordycepin | cordycepin | cordycepin | cordycepin |
| ARCA-Luc-SL | 1.0 ± 0.4 | 5.8 ± 2.0 | 4.0 ± 0.8 | 12.8 ± 1.0 |
| BTH-Luc-SL | 2.0 ± 1.2 | 11.4 ± 2.5 | 1.1 ± 0.6 | 18.9 ± 2.5 |
| ARCA-Luc-A₇₄ | 14.3 ± 2.6 | 9.0 ± 0.8 | 10.3 ± 3.5 | 25.4 ± 7.0 |
| BTH-Luc-A₇₄ | 10.6 ± 2.1 | 47.6 ± 2.5 | 10.1 ± 3.9 | 52.0 ± 4.4 |

^{a}Data are from FIGS. 1A-1B for Luc-SL mRNAs and FIGS. 5A-5B for Luc-A₇₄ mRNAs. Values represent the % of the initial mRNA introduced into cells remaining two hours after nucleoporation.

The novel technique can be used to produce an mRNA encoding essentially any protein of interest. The mRNA is more stable when introduced into cells, and therefore the mRNA yields a greater amount of the protein product because the mRNA is available to the translational machinery for a longer time. There are many proteins of high commercial interest that may be produced with the novel technique. One application of immediate therapeutic value is the synthesis of cancer antigens in dendritic cells in order to immunize a patient against the patient's own cancer. The dendritic cells then stimulate T-cells, to marshal the patient's own immune system against cancer cells. See, e.g., Kuhn A, Diken M, Kreiter S, Vallazza B, Tureci Ö, Sahin U. 2011. Determinants of intracellular RNA pharmacokinetics: Implications for RNA-based immunotherapeutics. RNA Biol 8: 35-43; Kuhn et al., 2010, Phosphorothioate cap analogs increase stability and translational efficiency of RNA vaccines in immature dendritic cells and induce superior immune responses in vivo. Gene Ther 17, 961-971.

When an mRNA is terminated at the 5′-end with an uncleavable cap and is also terminated at the 3′-end with cordycepin, it is more stable than an mRNA containing only the uncleavable cap (compare filled symbols in FIG. 1C to filled symbols in FIG. 1A). In our experiments the cordycepin modification was more effective in stabilizing mRNA than the uncleavable cap modification. Compare the filled symbols in FIG. 1A (cordycepin without uncleavable cap) to the open symbols in FIG. 1B (uncleavable cap without cordycepin). The greatest effect on stability was observed with both an uncleavable cap and a blocked 3′-terminus (FIG. 1B, filled symbols).

### FURTHER EXAMPLES

Examples of compositions and methods within the scope of the present invention include, but are not limited to, the following:

A method of synthesizing, in vitro or in vivo, an RNA molecule as described, said method comprising reacting ATP, CTP, UTP, and GTP, a chain-terminating nucleoside triphosphate as described, and a polynucleotide template in the presence of RNA polymerase, under conditions conductive to transcription by the RNA polymerase of the polynucleotide template into an RNA copy; whereby some of the RNA copies will incorporate the composition to make an RNA molecule as described, containing both an SL region and a chain-terminating nucleoside. For example, cordycepin may be incorporated at the 3′ terminus of the RNA molecule with yeast poly(A) polymerase (PAP). The same enzyme or other nucleotide polymerizing enzymes, e.g., RNA polymerase, may be used to incorporate other chain-terminating nucleosides. Alternatively, the chain-terminating nucleoside may be incorporated by chemical condensation using methods otherwise known in the art.

A method for synthesizing a protein or peptide in vitro, said method comprising translating an RNA molecule as described in a cell-free protein synthesis system, wherein the RNA molecule comprises an open reading frame, under conditions conductive to translating the open reading frame of the RNA molecule into the protein or peptide encoded by the open reading frame.

A method for synthesizing a protein or peptide in vivo or in cultured cells, said method comprising translating an RNA molecule as described in vivo or in cultured cells, wherein the RNA molecule comprises an open reading frame, under conditions conductive to translating the open reading frame of the RNA molecule into the protein or peptide encoded by the open reading frame.

A method as described, wherein the system is a native RNA translation system of a living organism, and wherein said method comprises the in vivo administration of the composition to the organism.

A method of synthesizing, in vitro or in vivo, an RNA molecule as described, said method comprising reacting ATP, CTP, UTP, GTP, and a polynucleotide template in the presence of RNA polymerase, under conditions conductive to transcription by the RNA polymerase of the polynucleotide template into an RNA copy, followed by the addition of a chain-terminating nucleoside at the 3′ end of the RNA; whereby some of the RNA copies will incorporate the composition to make an RNA molecule as described.

A method for synthesizing a protein or peptide in vivo or in cultured cells from an RNA molecule as described with a 3′ chain-terminating nucleoside, wherein said method synthesizes the protein or polypeptide in an amount that is at least 1.25 times, 1.5 times, 2 times, 3 times, 5 times, 8 times, 10 times, 15 times, or 20 times greater than would be synthesized by an otherwise-identical method using an otherwise-identical RNA molecule that lacked a 3′ chain-terminating nucleoside.

An RNA molecule as described, wherein the RNA molecule does not comprise a 3′ poly(A) tail; wherein a poly(A) tail is a tract that contains 10 or more, 15 or more, 20 or more, or 25 or more contiguous adenine residues without any intervening nucleosides other than adenine.

SL-containing mRNAs are expected to be more stable during S phase, so it is preferred (although not required) to use the novel method to produce proteins primarily during S phase in cultured cells. S-phase cells are the only cells that contain SLBP. Alternatively, one could modify the system to work during other phases of the cell cycle by adding an mRNA (either in the same molecule or a different molecule) that expresses SLBP. See, e.g., Sanchez and Marzluff, 2002, The stem-loop binding protein is required for efficient translation of histone mRNA in vivo and in vitro. Mol Cell Biol. 2002. 22(20):7093-104.

There are fundamental differences between the mechanism for translating poly(A)-containing mRNAs, and the mechanism for translating SL-containing mRNAs. (See the discussion in the “Background Art” section above.) The vast majority of mRNAs contain poly(A) rather than the histone SL. The cytoplasmic poly(A)-binding protein, PABP, is required for the translation of poly(A)-containing mRNAs but not for translating SL-containing mRNAs. This dependence could be exploited by using the invention to increase the production of proteins encoded by SL-containing mRNAs, namely, by selectively reducing poly(A)-dependent translation. For example, one of several possible ways to selectively decrease poly(A)-dependent translation without affecting SL-dependent translation would be to down-regulate intracellular levels of PABP with siRNA or miRNA. Another possibility would be to place the PABP gene under the control of a less efficient promoter. Still another possibility would be to overexpress Paip2 (an inhibitor of PABP); to do this transiently, one could transfect the cells with Paip2 mRNA. See Karim M M, Svitkin Y V, Kahvejian A, De Crescenzo G, Costa-Mattioli M, Sonenberg N. 2006. A mechanism of translational repression by competition of Paip2 with eIF4G for poly(A) binding protein (PABP) binding. PNAS 103: 9494-9499.

Most oncogene mRNAs are polyadenylated. For example, it has been reported that in myelomas and human T-cell leukemias, c-myc mRNA is stabilized and translated at a level seven times greater than the corresponding wild-type gene. See Hollis G F, Gazdar A F, Bertness V, Kirsch I R. 1988. Complex translocation disrupts c-myc regulation in a human plasma cell myeloma. Mol Cell Biol 8: 124-129. It could be beneficial to suppress the global translation levels of poly(A)-containing mRNA, while SL-containing anti-tumor mRNAs are expressed at unsuppressed levels. In summary, our invention allows the production in cells of higher quantities of a specific protein encoded by a synthetic RNA. By making the synthetic mRNA more stable, the translational machinery engages with the mRNA longer. Many mRNAs compete for available translational machinery. The mRNAs with higher translational efficiencies have an advantage over other mRNAs, and their protein products are relatively more abundant (all else being equal). By diminishing the intracellular levels or availability of PABP, our novel, synthetic, SL-containing mRNA has a translational advantage over all poly(A)-containing mRNAs generally. Therefore, more of the specific protein is produced.

Alternatively, the invention may also be used to stabilize microRNAs. MicroRNAs (miRNAs) can be used, for example, to silence a particular gene of interest. However, miRNAs are vulnerable to rapid degradation following transfection into cells. As is the case for SL-containing mRNAs, miRNAs undergo an oligouridylation-dependent breakdown pathway. The novel method may therefore also be used to stabilize these miRNAs, and thus to enhance RNA interference and the knock-down effect of these molecules. The novel method may also be used to stabilize any other type of RNA that undergoes a uridylation step to initiate degradation. For example, some polyadenylated mRNAs in S. pombe are also uridylated. It has been reported that miRNAs are oligouridylated in Arabidopsis thaliana, and that the oligouridylation triggers degradation of the miRNAs. See Li J, Yang Z, Yu B, Liu J, Chen X. 2005. Methylation protects miRNAs and siRNAs from a 3′-end uridylation activity in Arabidopsis. Current Biology 15: 1501-1507. Uridylation of pre-miRNAs in the cytoplasm prevents maturation by dicer, and results in the degradation immature products. See Heo I, Joo C, Kim Y-K, Ha M, Yoon M-J, Cho J, Yeom K-H, Han J, Kim V N. 2009. TUT4 in concert with Lin28 suppresses microRNA biogenesis through pre-microRNA uridylation. Cell 138: 696-708. It has been shown that miRNAs predominantly undergo 3′ non-template additions (NTA) of uridines and adenines in human, mouse and C. elegans. The particular 3′ NTA for specific miRNAs has been observed to change following differentiation of human embryonic stem cells, suggesting that post-transcriptional nucleotide addition is a physiologically regulated process in humans. See Wyman S K, Knouf E C, Parkin R K, Fritz B R, Lin D W, Dennis L M, Krouse M A, Webster P J, Tewari M. 2011. Post-transcriptional generation of miRNA variants by multiple nucleotidyl transferases contributes to miRNA transcriptome complexity. Genome Res 21: 1450-1461. Uridylation by the poly(U) polymerase Cid-1 of some polyadenylated mRNAs has been shown to stimulate their decapping in Schizosaccharomyces pombe. See Rissland O S, Norbury C J. 2009. Decapping is preceded by 3′ uridylation in a novel pathway of bulk mRNA turnover. Nat Struct Mol Biol 16: 616-623.

The complete disclosures of all references cited in this specification are hereby incorporated by reference, particularly including but not limited to the complete disclosures of the two priority applications. Also incorporated by reference are the complete disclosures of the following works by the inventors: W. Su et al., 2013, RNA 19:1-16; W. Su, Influence of the 5′-Terminal Cap and 3′-Terminal Structures on mRNA Stability, Translation, and Turnover in Mammalian Cells, PhD Dissertation (Louisiana State University Health Sciences Center, Shreveport, La. 2012); and W. Su, Influence of the 5′-Terminal Cap and 3′-Terminal Structures on mRNA Stability, Translation, and Turnover in Mammalian Cells (presentation given at Fred Hutchinson Cancer Center, Seattle, Wash., 2012); and R. Rhoads, Role of 5′ Decapping and 3′ Oligouridylation in Histone mRNA Turnover (presentation given at University of Texas Medical Center, Houston, Tex., 2012). In the event of an otherwise irreconcilable conflict, however, the present specification shall control.

What is claimed:

1. A synthetic RNA molecule that comprises the following consecutive elements (a) through (e) in the 5′ to 3′ direction:
(a) a modified or unmodified guanosine-derived cap;
(b) a 5′ untranslated region;
(c) a coding sequence having an open frame that encodes a protein or polypeptide;
(d) a 3′ untranslated region that terminates in a histone stem-loop; and
(e) a chain-terminating nucleoside at the 3′ end of the 3′ untranslated region that inhibits the addition of further nucleosides to said RNA molecule;
wherein said RNA molecule does not comprise a 3′ poly(A) tail; wherein a poly(A) tail is a tract that contains 15 or more contiguous adenine residues without any intervening nucleosides other than adenine.

2. The RNA molecule of claim 1, wherein said chain-terminating nucleoside is selected from the group consisting of 3′-deoxyadenosine (cordycepin); 3′-deoxyuridine; 3′-deoxycytosine; 3′-deoxyguanosine; 3′-deoxythymine; 2′,3′-dideoxyadenosine; 2′,3′-dideoxyuridine; 2′,3′-dideoxycytosine; 2′,3′-dideoxyguanosine; 2′,3′-dideoxythymine; a 2′-deoxynucleoside; a 2′-O-methylnucleoside; a 3′-O-methylnucleoside; a 3′-O-ethylnucleosides; and a 3′-arabinoside.

3. The RNA molecule of claim 1, wherein said chain-terminating nucleoside is 3′-deoxyadenosine (cordycepin).

4. The RNA molecule of claim 1, wherein said guanosine-derived cap is 7-methylguanosine.

5. The RNA molecule of claim 1, wherein said guanosine-derived cap is an anti-reverse cap analog.

6. The RNA molecule of claim 1, wherein said guanosine-derived cap is a borano-two-headed cap analog.

7. A method for synthesizing a protein or polypeptide in vitro or in cultured cells, said method comprising introducing into cultured cells or into a cell-free protein synthesis system the RNA molecule of claim 1, under conditions conducive to translating the open reading frame into the protein or polypeptide.

8. The method of claim 7, wherein said method is conducted in vitro in a cell-free protein synthesis system.

9. The method of claim 7, wherein said method is conducted in cultured cells.

10. The method of claim 7, wherein the chain-terminating nucleoside is selected from the group consisting of 3′-deoxyadenosine (cordycepin); 3′-deoxyuridine; 3′-deoxycytosine; 3′-deoxyguanosine; 3′-deoxythymine; 2′,3′-dideoxyadenosine; 2′,3′-dideoxyuridine; 2′,3′-dideoxycytosine; 2′,3′-dideoxyguanosine; 2′,3′-dideoxythymine; a 2′-deoxynucleoside; a 2′-O-methylnucleoside; a 3′-O-methylnucleoside; a 3′-O-ethylnucleosides; and a 3′-arabinoside.

11. The method of claim 7, wherein the chain-terminating nucleoside is 3′-deoxyadenosine (cordycepin).

12. The method of claim 7, wherein said method synthesizes the protein or polypeptide in an amount at least twice as much as would be synthesized by an otherwise-identical method using an otherwise-identical RNA molecule that lacked a 3′ chain-terminating nucleoside.

13. The method of claim 7, wherein the guanosine-derived cap is 7-methylguanosine.

14. The method of claim 7, wherein the guanosine-derived cap is an anti-reverse cap analog.

15. The method of claim 7, wherein the guanosine-derived cap is a borano-two-headed cap analog.


SEQUENCE LISTING (17 sequences)

SEQ ID NO: 1 - DNA, 19 bp, Artificial sequence
  Synthetic PCR primer
ggatggaaccgctggagag

SEQ ID NO: 2 - DNA, 23 bp, Artificial sequence
  Synthetic PCR primer.
gcatacgacgattctgtgatttg

SEQ ID NO: 3 - DNA, 24 bp, Artificial sequence
  Synthetic PCR primer.
atcgtggattacgtcgccagtcaa

SEQ ID NO: 4 - DNA, 24 bp, Artificial sequence
  Synthetic PCR primer
tttccgcccttcttggcctttatg

SEQ ID NO: 5 - DNA, 17 bp, Artificial sequence
  Synthetic PCR primer
cgagccgcctggatacc

SEQ ID NO: 6 - DNA, 25 bp, Artificial sequence
  Synthetic PCR primer
cagttccgaaaaccaacaaaataga

SEQ ID NO: 7 - DNA, 21 bp, Homo sapiens
caaaggtcttttcagagccac

SEQ ID NO: 8 - RNA, 26 nt, Homo sapiens
ccaaaggcucuuuucagagccaccca

SEQ ID NO: 9 - RNA, 25 nt, Drosophila melanogaster
ccaaaaguccuuuucagggcuacca

SEQ ID NO: 10 - RNA, 26 nt, Caenorhabditis elegans
ccaacggcccucuuuagggccacaaa

SEQ ID NO: 11 - RNA, 26 nt, Dictyostelium discoideum
ccaaagguguuuuuuaacaccacaca

SEQ ID NO: 12 - RNA, 26 nt, Trichomonas vaginalis
uaauuggauuuuuucaaauccaccug

SEQ ID NO: 13 - RNA, 26 nt, Plasmodium berghei
uuugaggcuucccuagaagccauuac

SEQ ID NO: 14 - RNA, 26 nt, Tetrahymena thermophila
guuacagguauauuuaucccuacuaa

SEQ ID NO: 15 - RNA, 26 nt, Mus musculus
ccaacggcucuuuucagagccaccca

SEQ ID NO: 16 - RNA, 26 nt, Volvox carteri
aacccgguguuuuuuaacaccaccgc

SEQ ID NO: 17 - RNA, 26 nt, Chlamydomonas reinhardtii
aacucgguguuucucaacaccaccua